(19) United States
(12) Patent Application Publication
(10) Pub. No.: US 2022/0386559 A1
(43) Pub. Date: Dec. 8, 2022

(54) REMINISCENCE THERAPY AND MEDIA SHARING PLATFORM

(71) Applicant: Telememory Holding Corporation, Kansas City, KS (US)

(72) Inventors: David KEENE, San Diego, CA (US); Edward Michael COX, San Diego, CA (US); Yevgeniy KOSTIKOV, San Diego, CA (US); Martin CULJAT, San Diego, CA (US); Rebecca Emily SENGER, San Diego, CA (US); Melissa GUANZON, San Diego, CA (US); Brian John KANG, San Diego, CA (US); Michael STANGE, San Diego, CA (US)

(21) Appl. No.: 17/585,398

(22) Filed: Jan. 26, 2022

Related U.S. Application Data
(63) Continuation of application No. 17/253,091, filed on Dec. 16, 2020, now abandoned.
(60) Provisional application No. 62/688,269, filed on Jun. 21, 2018.

(51) Int. Cl. A01K 1/06; A01K 1/00; G16H 40/67; G16H 20/70; G16H 80/00; G06N 20/00; G06V 40/16

(52) U.S. Cl. CPC A01K 1/0613; A01K 1/0035; G16H 40/67; G16H 20/70; G16H 80/00; G06N 20/00; G06V 40/174

(57) ABSTRACT

Described are reminiscence therapy and media sharing platform, methods, and systems for a patient user which provide an immediate and First Mobile Application positive impact on emotional functioning inpatients with dementia, major neurocognitive disorders, social isolation, traumatic brain injury (TBI), and psychiatric conditions such as posttraumatic stress disorder, by reducing anxiety, depression, and overall emotional distress.

### CROSS-REFERENCE

This application is a Continuation of U.S. patent application Ser. No. 17/253,091, filed Dec. 16, 2020, which is a U.S. National Phase Application under 35 U.S.C. § U.S.C. § 371 of International Application No. PCT/US/2019/038568, filed Jun. 21, 2019, which claims the benefit of U.S. Provisional Application No. 62/688,269, filed Jun. 21, 2018, which is hereby incorporated by reference in its entirety herein.

### BACKGROUND OF THE INVENTION

The U.S. population is rapidly aging, with 21% of the population, or 74 million people, expected to be older than 65 by 2030. Social isolation is estimated to impact up to 50% of seniors, and loneliness has been shown to affect approximately one-third of adults later in life. Social isolation and loneliness can have a significant impact on health and quality of life, with health risks comparable to traditional clinical risk factors, such as smoking, obesity, high blood pressure, and high cholesterol. Prolonged isolation has been estimated by A ARP (formerly American Association of Retired Persons) to be equivalent to smoking 15 cigarettes a day, and in another study it was shown to increase the likelihood of mortality by more than 25%.

Social isolation in older adults has been associated with decreased resistance to infection, cognitive decline and mental health conditions such as depression and dementia, increased numbers of falls, increased emergency department admissions, longer hospital stays and delayed discharges, increased drinking and smoking, sedentary lifestyle, and poor nutrition. Unfortunately, there are no interventions that have been shown to be effective in reducing social isolation or loneliness on a large scale.

According to the Alzheimer's Association, Alzheimer's disease (AD) is the “only top 10 cause of death that cannot be prevented, cured or even slowed.” There are about 5.7 million Americans living with Alzheimer's and other forms of dementia today, and that number is projected to rise to 14 million by 2050. As a result, the cost of care for dementia patients is expected to increase from $277 billion in 2018 to more than $1.1 trillion by 2050. Unfortunately, there are only four FDA-approved Alzheimer's drugs, and all provide moderate and short-term benefits. Further, Alzheimer's drugs under development have had a 99.3% failure rate thereby leaving few therapeutic options on the horizon.

A more recent focus for pharmaceutical companies and organizations such as the Alzheimer's Association has been on addressing the behavioral symptoms of Alzheimer's disease. These symptoms, including agitation, depression, anxiety, and apathy take a toll on quality of life. add burden to family caregivers and can increase the overall cost of care. Additionally, the behavioral symptoms of are often the primary reason behind the transition from home care to long term assisted living facilities, rather than the disease itself. This is compounded with widespread “off-label” use of antipsychotic medications in this patient population despite their lack of effectiveness and FDA “black box warnings” of double the risk of death when they are used in seniors with dementia.

Reminiscence Therapy (RT) is an evidence-based behavioral intervention that involves the introduction of familiar pictures, music, or other materials to help individuals reminisce about their past experiences. RT is the most commonly used non-pharmacological therapy in Alzheimer's and other types of dementia, and has been used since the 1960s in home care and in the nursing home and hospital settings. Published reviews of randomized, controlled clinical trials using RT have suggested that RT can significantly reduce behavioral symptoms in individuals with dementia and in seniors with social isolation.

A major limitation of RT is that it must be provided physically by a human caregiver. For this reason, it is highly labor-intensive, repetitive, and time-consuming, and, therefore, challenging to deliver at scale. It is simply not practical for a family caregiver to sit with a dementia patient to go over the same photo scrapbook, home videos, or music on a daily basis, and using a therapist or professional therapist for frequent RT is cost prohibitive. Therefore, RT is usually given sporadically and in formal group therapy sessions. Furthermore, RT is given when it is convenient to the caregiver, not at the convenience of the patient, further limiting the consistency of its use. There is a need for digital RT technologies that are scalable and that can be provided frequently, consistently, and without the need to depend on family members or healthcare providers.

### SUMMARY OF THE INVENTION

Although Reminiscence Therapy (RT) has been shown to be effective for treatment of AD, other dementias, and social isolation without medicinal side effects, current means of performing (RT) are highly labor-intensive. Individual RT is generally recommended for its effectiveness, but can be very time consuming for the caregiver and expensive to perform. These current constraints greatly limit the ability for most patients to receive consistent, daily RT therapy as recommended. A technology platform that digitizes RT has the potential to deliver RT to these individuals frequently, effectively, and at scale.

Seniors who are socially isolated can also benefit from RT. Many seniors are unable to utilize new technologies, such as mobile devices, tablets, and computers, and therefore are not able to access communication tools and social media platforms. Because families are spread across the country and have busy lives, it is often easy for seniors to get left out if they can't adapt to new technologies. This can lead to limited engagement with family and friends and result in loneliness and social isolation. Therefore, they can also benefit from technology platforms that are simple to use, and that deliver RT content as well as more recent content that updates them on current family events and engages them socially with their family and friends.

A technology platform that delivers photo, video, and audio content such as RT can further he used in other scenarios, such as for patients recovering from stroke or patients undergoing chemotherapy. The patients can be given messages and content from their family members, who can become more motivated because they feel that others are also involved in their care and in their, journey to recovery. Customized content and educational material can also be provided on the tablet to the patient. The same technology platform can also help to educate or engage the patient's loved ones, or enable the physician to remotely monitor the patients progress.

Digital therapeutics is a new subsection of digital health that strives to directly deliver a cognitive behavioral therapy (CBT) via use or interaction with software technology, typically using smart phones or, in our case, using a dedicated computer tablet. The goal of digital therapeutics is to mirror an effective CBT already in use, but to do so using technology in order to scale up for a larger patient population. This adoption of technology amplifies care, improving patients' behavior or functioning while reducing the cost of care.

A digital therapeutic approach to RT is proposed herein that allows family members to share memories win their loved ones by uploading old photos from their mobile device and narrating stories over the photos. The platform transforms the short audio notes and individual photos into rich documentary-like stories that are then archived in a private and secured database. Videos and music can also be shared and combined into stories. These stories can then be viewed easily by the patient on a customized computer tablet with a user interface that is simple to operate, even for seniors with Alzheimer's disease or dementia.

The systems, platforms, and methods herein can provide a positive impact on functioning of people suffering from dementia or social isolation, or people recovering from conditions such as stroke or diseases such as cancer, or any number of other conditions or diseases. Further, the systems, platforms, and methods herein can reduce caregiver and family burden by enabling family members to remotely engage with the patient to assist in the therapy and recovery of their loved ones from anywhere in the world and on their own time.

The online-based story-sharing platform, systems, and methods herein allow users to record audio or submit written descriptions or comments related to photos or videos to easily share memories with family members, wherein the family member may be suffering from a neurological or psychiatric condition. As such, multiple family members from around the world and at different times may collaborate to form meaningful and therapeutic the stories in just a few minutes a day. The platform transforms the media and descriptions received by family members into rich documentary-like stories that may be archived in a private and secured database. These stories can then be viewed easily through a simple user interface via any media device whenever the patient chooses. Thus, RT patients can reminisce about their past in a structured or unstructured time or facility, without the necessity for one-on-one administration.

Further, the online-based story-sharing platform, systems, and methods herein are configured to allow users to record audio or submit written descriptions or comments related to photos or videos for media sharing. Further, the online-based story-sharing platform, systems, and methods herein are configured to enable sharing of educational media. Such platforms ensure that shared media is substantial, meaningful, and relevant to both the user sharing and receiving the media

One aspect provided herein is a reminiscence therapy and media sharing platform for a patient user comprising: a contributing user mobile processor configured to provide a first mobile application comprising: a prompt module receiving and displaying a request for a first media from a contributing user; a first media module receiving the first media from the contributing user; and a server processor configured to provide a server application comprising: a request module generating the request and submitting the request to the prompt module; and a chronicle module receiving the first media and generating a story based on the first media; and a patient user mobile processor configured to provide a second mobile application comprising: a communications module receiving the story; a media output module presenting the story to the patient user; a reaction module measuring a reaction of the patient user while media output module presents the story to a patient user; and a feedback module transmitting the reaction to the request module; wherein the request module generates a subsequent request based on the reaction.

In some embodiments at least one the first media and the story comprises a photograph, a video, an image, a gif, an emoji, a text, an audio recording, music, or any combination thereof. In some embodiments the request comprises a media type, a media theme, a media subject, a media color, a media date, a media duration, or any combination thereof. In some embodiments the prompt module displays the request for the first media via a screen, a speakerphone, a phone call, a text message, a push notification, an email, or any combination thereof. In some embodiments the reaction comprises an anger parameter, a contempt parameter, a fear parameter, a happiness parameter, a surprise parameter, a sadness parameter, an attention parameter, a symmetry parameter, an eye quadrant, an eve fixation time, an eye fixation duration, a button press, or any combination thereof. In some embodiments the reaction does not comprise a button press. In some embodiments the reaction module measures the reaction of the patient user through a facial expression recognition process. In some embodiments the facial expression recognition process is configured for facial expression recognition of the patient user having an age of greater than about 50 years. In some embodiments the facial expression recognition process comprises a computer learning process. In some embodiments the contributing user comprises a plurality of contributing users. In some embodiments the chronicle module generates the story by performing at least the following: performing a facial expression recognition on the first media; performing object recognition on the first media; determining a geographic location associated with the first media. In some embodiments the facial expression recognition process comprises age recognition, sex recognition, environment recognition, object recognition, or any combination thereof. In some embodiments the chronicle module further stores the story. In some embodiments, the server application further comprises a database storing a plurality of the templates. In some embodiments the first mobile application further comprises a descriptor module notifying the contributing user to submit a second media based on the first media and a template. In some embodiments the second media is further based on the third media. In some embodiments the first mobile application further comprises a second media module receiving the second media from the contributing user. In some embodiments the chronicle module further generates the story based on the second media. In some embodiments the second media comprises a photograph, a video, an image, a gif, an emoji a text, or any combination thereof. In some embodiments the server application further comprises a media agglomeration module determining, and receiving a third media from a third media source. In some embodiments the third media source comprises a social media image, a social media text, a social media video, a public media image, a public media text, a public media video, or any combination thereof. In some embodiments the chronicle module further generates the story based on the third media. In some embodiments the media agglomeration module determines the third media by a computer learning process. In some embodiments the prompt module receives the request through a cellular network, a wireless network, a Bluetooth signal, a wired signal, or any combination thereof. In some embodiments the first media module receives the first media through a cellular network, a wireless network, a Bluetooth signal, a wired signal, or any combination thereof. In some embodiments the chronicle module receives the first media through a cellular network, a wireless network, a Bluetooth signal, a wired signal, or any combination thereof. In some embodiments the communication module receives the story through a cellular network, a wireless network, a Bluetooth signal, a wired signal, or any combination thereof. In some embodiments the feedback module transmits the reaction through a cellular network, a wireless network, a Bluetooth signal, a wired signal, or any combination thereof.

Another aspect provided herein is a reminiscence therapy and media sharing platform for a patient user comprising: a contributing user mobile processor configured to provide a first mobile application comprising: a prompt module receiving and displaying a request for a first media from a contributing user; a first media module receiving the first media from the contributing user; a descriptor module notifying the contributing user to submit a second media based on the first media and a template; and a second media module receiving the second media from the contributing user; a server processor configured to provide a server application comprising: a database storing a plurality of the templates; a request module generating and submitting the request; and a chronicle module receiving the first media and the second media and generating a story based at least on the first media and a second media; and a patient user mobile processor configured to provide a second mobile application comprising: a communications module receiving the story; a media output module presenting the story to the patient user; a reaction module measuring a reaction of the patient user while media output module presents the story to a patient user; and a feedback module transmitting the reaction to the request module; wherein the request module generates the request based on the reaction.

Another aspect provided herein is a reminiscence therapy and media sharing platform for a patient user comprising: a contributing user mobile processor configured to provide a first mobile application comprising a first media module receiving a first media from a contributing user; and a server processor configured to provide a server application comprising a chronicle module receiving the first media and generating a story based on the first media; and a patient user mobile processor configured to provide a second mobile application comprising: a communications module receiving the story; a media output module presenting the story to the patient user; a reaction module measuring a reaction of the patient user while the media output module presents the story to a patient user; and a feedback module transmitting the reaction to the request module; wherein the server processor further generates a subsequent request based on the reaction.

In some embodiments, at least one the first media and the story comprises a photograph, a video, an image, a text, or any combination thereof. In some embodiments, the reaction comprises an anger parameter, a contempt parameter, a fear parameter, a happiness parameter, a surprise parameter, a sadness parameter, a symmetry parameter, an eve quadrant, an eye fixation time, an eye fixation duration, a button press, or any combination thereof. In some embodiments, the reaction does not comprise a button press. In some embodiments, the reaction module measures the reaction of the patient user through a facial expression recognition process. In some embodiments, the facial expression recognition process is configured for facial recognition of the patient user having an age of greater than about 50 years. In some embodiments, the facial expression recognition process comprises a computer learning process. In some embodiments, the contributing user comprises a plurality of contributing users. In some embodiments, the chronicle module generates the story by performing at least the following: performing a facial recognition on the first media; performing an object recognition on the first media; determining a geographic location associated with the first media. In some embodiments, the facial expression recognition process comprises an age recognition, a sex recognition, an environment recognition, an object recognition, or any combination thereof. In some embodiments, the chronicle module further stores the story. In some embodiments, the server application further comprises a database storing a plurality of the templates. In some embodiments, the first mobile application further comprises a descriptor module notifying the contributing user to submit a second media based on the first media and a template. In some embodiments, the second media is further based on the third media. In some embodiments, the first mobile application further a second media module receiving the second media from the contributing user. In some embodiments, the chronicle module further generates the story based on the second media. In some embodiments, the second media comprises a photograph, a video, an image, a gif, an emoji, a text, or any combination thereof. In some embodiments, the server application further comprises a media agglomeration module determining and receiving a third media from a third media source. In some embodiments, the third media source comprises a social media image, a social media text, a social media video, a public media image, a public media text, a public media video, or any combination thereof. In some embodiments, the chronicle module further generates the story based on the third media. In some embodiments, the media agglomeration module determines the third media by a computer learning process. In some embodiments, the first media module receives the first media through a cellular network, a wireless network, a Bluetooth signal, a wired signal, or any combination thereof. In some embodiments, the chronicle module receives the first media through a cellular network, a wireless network, a Bluetooth signal, a wired signal, or any combination thereof. In some embodiments, the communication module receives the story through a cellular network, a wireless network, a Bluetooth signal, a wired signal, or any combination thereof. In some embodiments, the feedback module transmits the reaction through a cellular network, a wireless network, a Bluetooth signal, a wired signal, or any combination thereof.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a schematic diagram of a first exemplary reminiscence therapy and media sharing platform for a patient user;

FIG. 2 shows a schematic diagram of a second exemplary reminiscence therapy and media sharing platform for a patient user;

FIG. 3 shows a schematic diagram of a third exemplary reminiscence therapy and media sharing platform for a patient user;

FIG. 4 shows an exemplary contributing user device and an exemplary patient user device;

FIG. 5 shows an exemplary patient user device and a patient user;

FIG. 6 shows an exemplary patient user device and a story;

FIG. 7 shows an exemplary reaction of the patient user;

FIG. 8 shows an exemplary image of a first mobile application;

FIG. 9 shows another exemplary image of a first mobile application;

FIG. 10 shows another exemplary image of a first mobile application;

FIG. 11 shows another exemplary image of a first mobile application;

FIG. 12 shows another exemplary image of a first mobile application;

FIG. 13 shows another exemplary image of a first mobile application;

FIG. 14 shows another exemplary image of a first mobile application;

FIG. 15 shows an exemplary image of a clarification;

FIGS. 16A-16E show patient user evaluation metrics before and idler media rehabilitation treatment;

FIG. 16A shows exemplary measurement results of an emotional thermometer (ET) test;

FIG. 16B shows exemplary measurement results of a State Anxiety Inventory (STAI) test;

FIG. 16C shows exemplary measurement results of a Hospital Anxiety and Depression Scale (HADS) test;

FIG. 16D shows exemplary measurement results of a Neuro-Quality of Life (NQOL) test.

FIG. 16E shows exemplary measurement results of a Caregiver Questionnaire (CQ) test;

FIG. 17 shows the effect sizes for the exemplary measurements in FIGS. 16A-16E;

FIG. 18 shows a schematic diagram of an exemplary digital processing device; in this case, a device with one or more CPUs, a memory, a communication interface, and a display;

FIG. 19 shows a schematic diagram of an exemplary web/mobile application provision system; in this case, a system providing browser-based and/or native mobile user interfaces;

FIG. 20 shows a schematic diagram of an exemplary cloud-based web/mobile application provision system; in this case, a system comprising an elastically load balanced, auto-scaling web server and application server resources as well synchronously replicated databases;

FIG. 21 shows the artificial intelligence chatbot texting interface with a contributor on a mobile user interface; and

FIG. 22 shows the steps to create a closed feedback system for therapeutic use.

### DESCRIPTION OF THE INVENTION

The platforms, systems, and methods personalizes the Reminiscence Therapy (RT) or the story, media, media and education sharing for each patient or customer through custom stories, and optimized content through a feedback mechanism, a machine learning algorithm, and a custom emotional recognition software. As such, the disclosure herein allows for an optimal, low-cost, scalable, automated, and personalized reminiscence therapy and for communal story and educational sharing. Further, the use of family engagement herein benefits the caregiver as well as the patient. Finally, the disclosure herein can be implemented at any home or senior care/memory care facility, in urban or in rural locations, may not require a dedicated kiosk, equipment, or training, and can be used by patients and caregivers of all ages and technical skills. Reminiscence therapy platform for a patient user

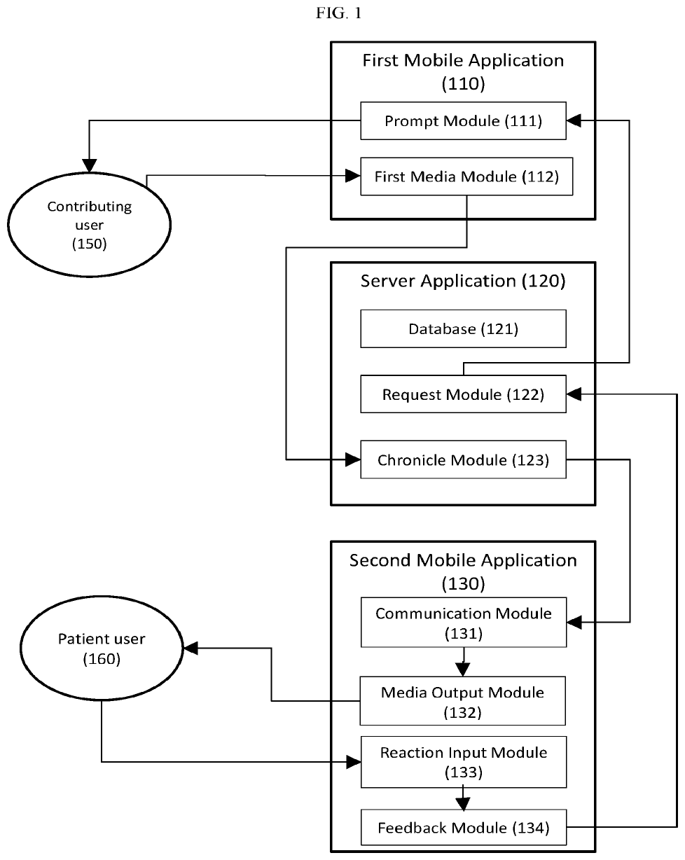

FIG. 1 shows a schematic diagram of a first exemplary reminiscence therapy and media sharing platform for a patient user comprising a contributing user mobile processor configured to provide a first mobile application, a server processor configured to provide a server application 120, and a patient user mobile processor configured to provide a second mobile application 130. In some embodiments, the patient user comprises a patient. In some embodiments, the contributing user comprises a family member, caregiver, or a friend.

In some embodiments, the first mobile application 110 comprises a prompt module 111, and a first media module 112. In some embodiments, the prompt module 111 receives and displays a request fora first media from a contributing user 150. In some embodiments the prompt module 111 displays the request for the first media via a screen, a speakerphone, a phone call, a text message, a push notification, an email, or any combination thereof. In some embodiments, the first media module 112 receives the first media from the contributing user 150. In some embodiments the contributing user 150 comprises a plurality of contributing users 150. In some embodiments the first mobile application 110 further comprises a descriptor module notifying the contributing user 150 to submit a second media based on the first media and a template. In some embodiments the second media is further based on the third media. In some embodiments the first mobile application 110 further comprises a second media module receiving the second media from the contributing user 150

In some embodiments, the server processor is configured to provide a server application 120 comprising a request module 122 and a chronicle module 123. In some embodiments the request module 122 generates the request and submits the request to the prompt module 111. In some embodiments, the request module 122 generates the request based on the reaction. In some embodiments, the chronicle module 123 receives the first media and generating a story based on the first media. In some embodiments the chronicle module 123 generating the story comprises: performing a facial recognition on the first media; performing object recognition on the first media; determining a geographic location associated with the first media; or any combination thereof. In some embodiments the chronicle module 123 further stores the story.

In some embodiments, the server application 120 further comprises a database 121 storing a plurality of the templates. In some embodiments the server application 120 further comprises a media agglomeration module determining and receiving a third media from a third media source. In some embodiments the third media source comprises a social media image, a social media text, a social media video, a public media image, a public media text, a public media video, or any combination thereof. In some embodiments the chronicle module 123further generates the story based on the third media. In some embodiments the media agglomeration module determines the third media by a computer learning process.

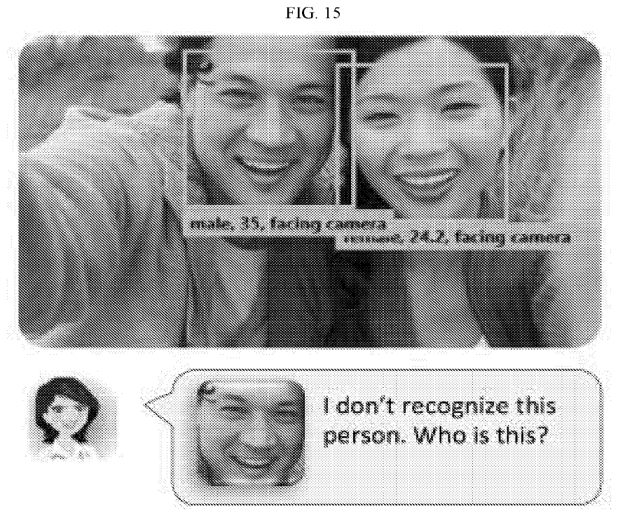

In some embodiments at least one of the chronicle module 123 and the first media module 112 further sends a clarification to the prompt module 111, wherein the clarification is based on the first media. In some embodiments at least one of the chronicle module 123 and the first media module 112 employs a facial expression recognition process to formulate the clarification. In some embodiments the clarification comprises a request for an event, a time, a person, a place, or any alternative information associated with the media. In some embodiments the clarification is based on the theme, the reaction, or both. In some embodiments the clarification prevents confusion and provides more useful therapeutic information to the patient user. In one example, per FIG. 15. the first media comprises an image of two people, whereby the facial expression recognition process determines that the person to the right is a known contributing user, and whereby the facial expression recognition process recognizes a second person in the picture, but does not know the identity of the second person. As such, the clarification may include a request for the identification of the second person, whereby the contributing user may respond with a message that “this is my new boyfriend Tom.” The platform may then recognize and provide indications of any future media comprising Tom.

In some embodiments, the patient user mobile processor is configured to provide a second mobile application 130 comprising a communications module, a reaction module 133, and a feedback module 134. In some embodiments. The communication module 131 receives the story. In some embodiments, the media output module 132 presents the story to a patient user 160. In some embodiments, the reaction module 133 measures a reaction of the patient user 160 while media output module 132 presents the story to a patient user 160. In some embodiments the feedback module 134 transmits the reaction to the request module 122. In some embodiments the feedback module 134 further transmits the reaction to the request first mobile application 210.

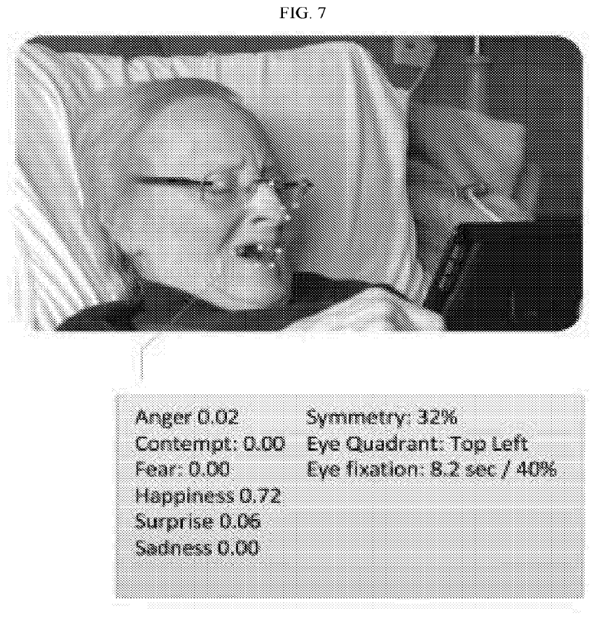

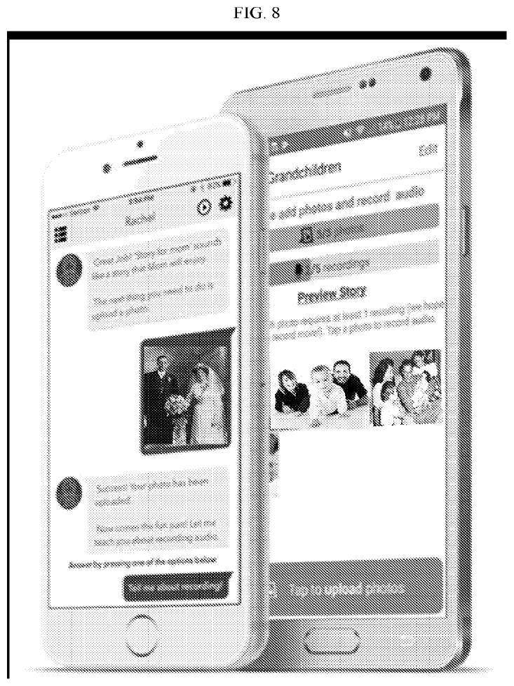

In some embodiments the reaction comprises an anger parameter, a contempt parameter. a fear parameter, a happiness parameter, a surprise parameter, a sadness parameter, a symmetry parameter, an eye quadrant, an eye fixation time, an eye fixation duration, a button press, or any combination thereof. FIG. 7 shows an exemplary reaction of the patient user. In some embodiments the reaction does not comprise a button press. In some embodiments the reaction module 133 measures the reaction of the patient user 160 through an auditory or visual confirmation. An example of a confirmation comprises the second mobile application 230 emitting a question regarding the story, such as “do you like this.” The second mobile application 230 may then record an auditory or tactile response to the auditory confirmation.

In some embodiments the reaction module 133 measures the reaction of the patient user 160 through a facial expression recognition process. In some embodiments the facial expression recognition process is configured for facial expression recognition of the patient user 160 having an age of greater than about 50 years. In some embodiments the facial expression recognition process comprises a computer learning process. In some embodiments, the facial expression recognition process enables the determination of the patient's feelings, likes, dislikes, fears, attention, and reminiscence towards a specific media, a type of media, the subject of a media, or any combination thereof. Emotions may be monitored by tracking facial landmarks or other computer vision techniques and comparing the data to known emotional visual data (for example, comparing tracked landmarked locations to commercially available databases e.g., MultiPlE, MM1, CK+, DISFA, FERA, SFEW, FER2013).

In some embodiments, the facial expression recognition process is further configured to measure a physiological response of the patient user 160 comprising a heart rate, a blood pressure, an eye focus, an eye movement, a head movement, voice or other audible feedback, or any combination thereof.

The platform may personalize the facial expression recognition process by forming a baseline for each patient user 160. The baseline may correlate to each user's 160 ability and means of forming facial expressions in response to certain emotions. The emotion may be determined by calculating a Rostov Scale Rating, wherein a positive emotion correlates to a high Rostov Scale Rating, and wherein a negative emotion correlates to low Rostov Scale Rating. This baseline may be updated and improved as the patient user 160 ages and/or experiences different emotions. In addition to quantifying the reaction of a patient user 160, the Rostov Scale Rating may be employed to elicit response from the patient user 160 to medical, legal, or clinical trial questionnaires.

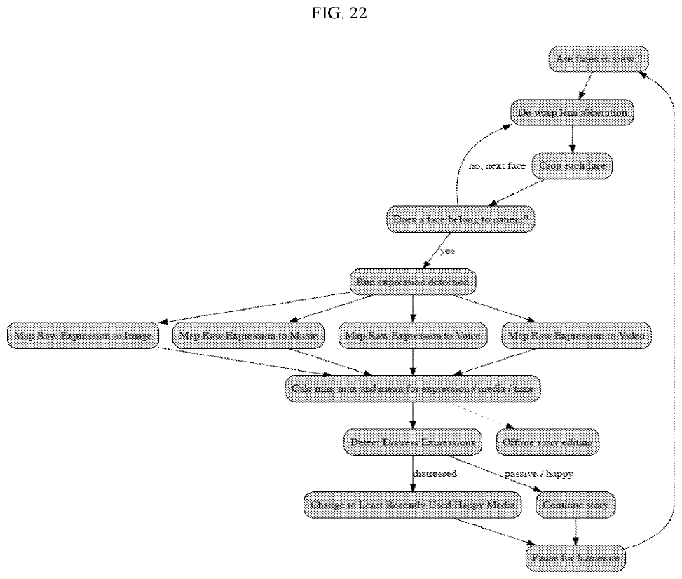

FIG 22. describes the steps to create a closed feedback system for therapeutic use. The system first analyses the captured video and or audio stream to detect if a face is in the frame. This detection system is tailored to the lens that is capturing the images, so a face can be tested even if it is warped by the lens. If a face is detected, for each face, the image is dewarped (i.e., the lens aberration is removed) and is cropped out of the large stream image. If multiple faces arc discovered in the prior step, it may be more efficient to dewarp the couple image and crop faces out in the second step. If there is only a single face detected in the prior step, it may be more efficient to crop first and then dewarp just the cropped image in the second step. For each image, a face recognition algorithm is performed on the face and there is an attempt to match the face to known patients who use this device. If the face belongs to the patient, the expression detection algorithm is run on that face. This algorithm uses computer vision to compare features of the face to a collection of known expressions and give a rating of various expressions that match the submitted face. The expression information is then mapped to the current image or video on screen, music playing and other audio playing. The expression information is then mapped to information discovered by image and video analysis of the displayed media (e.g., landmarks, subjects of media, people present in media, colors in v). The expression information is mapped to information discovered by audio analysis of the audio playing (e.g; key, volume, tone of voice, who is speaking). Negative emotions can be detected in facial expressions. In this case, media is marked that consistently elicits negative expressions for possible removal. All media is sent to expression mapping back to the server for improved communication with the family to encourage them to send more content, to reorder media for the next therapeutic video, to add additional images that are similar in subject and meta analysis to the high ranking media, or other purpose.

In one example, the image of the user's face is processed via a convolutional network with feature-based cascade classifiers (eg: Haar Cascades) for face detection. Secondly, the facial expression detection technology may then be trained and implemented for specific emotion detection using the Multi-PlE Face Database (337 adult subjects/200, 000+ images), available through a commercial license. Alternatively, the facial expression detection technology could be trained on a custom set of images for the senior face.

Priming the facial expression recognition process to function properly when analyzing elderly faces is of utmost importance, as such cognitive disorders rue often associated with age. The appearance of the face typically changes with age, including wrinkles, loss of underlying fat, loss of muscle tone, thinning skin, dark spots of skin pigment, missing teeth, receding gums, shrunken lips, loss of jaw bone mass, lengthening nose and ears, bags under eyes, drooping eyelids, changes in the outer cornea, and changing color of the iris. Facial expression databases, however, are not available for elderly patients. As such, the image recognition may be programed by recording a plurality of test subjects, each performing a plurality of facial expressions in a variety of lighting conditions. The facial expressions may comprise happiness, sadness, surprise, anger, disgust, fear, and a neutral expression. Vectors and points from facial landmarks may then be collected and plotted in graphs of emotion vs time (in milliseconds) to distinguish each pose.

In one example, the facial expression recognition process may detect that the patient user responds positively to pictures of dogs, a specific family member, or pictures of weddings, and negatively to pictures of the beach, male voices, and fast pace transitions. As such, the platform can prioritize content with positive responses and re-edit stories for the patient user.

In some embodiments, the feedback of the reaction enables a determination of a like or preference of the patient user. The reaction may comprise an image of the patient user, a play statistic, or an emotional statistic. The play statistic may comprise play duration, a play count, or both. The emotional statistic may comprise a feeling, an eye movement, a smile indicator, level of attention, or any combination thereof. In some embodiments, the like or preference or attention, enables the optimized selection and/or organization of the media for maximal therapeutic effect. This reaction can be attained from the patient user, the contributing user, or both. Further, such a feedback reaction allows the contributing user to receive an indication regarding the wellbeing, temperament, and/or status of the patient user. Also, the reaction may encourage the contributing user to continue to send more and specific types/genres/subjects of media to act as an effective reward system. In one example, if the reaction includes a picture of a patient smiling in response to a picture of her dog, the family is encouraged to provide more such content with the knowledge that the reminiscence therapy is helping to improve the patient's quality of life. Thus the feedback reaction ensures that the patient is being treated with content of positive therapeutic value.

In some embodiments, at least one, the media contained in the story comprises a photograph, a video, an image, a gif, an emoji, a text, audio narration, music, or any combination thereof. In some embodiments the request comprises a media type, a media theme, a media subject, a media color, a media date, a media duration, or any combination thereof. In some embodiments the facial expression recognition process comprises an age recognition, a sex recognition, an environment recognition, an object recognition, or any combination thereof. In some embodiments the chronicle module 123 further generates the story based on the second media. In some embodiments the second media comprises a photograph, a video, an image, a gif, an emoji, a text, or any combination thereof.

In some embodiments the prompt module 111 receives the request through a cellular network, a wireless network, a Bluetooth signal, a wired signal, or any combination thereof. In some embodiments the first media module 112 receives the first media through a cellular network, a wireless network, a Bluetooth signal, a wired signal, or any combination thereof. In some embodiments the chronicle module 123 receives the first media through a cellular network, a wireless network, a Bluetooth signal, a wired signal, or any combination thereof. In some embodiments the communication module 131 receives die story through a cellular network, a wireless network, a Bluetooth signal, a wired signal, or any combination thereof. In some embodiments the feedback module 134 transmits the reaction through a cellular network, a wireless network, a Bluetooth signal, a wired signal, or any combination thereof.

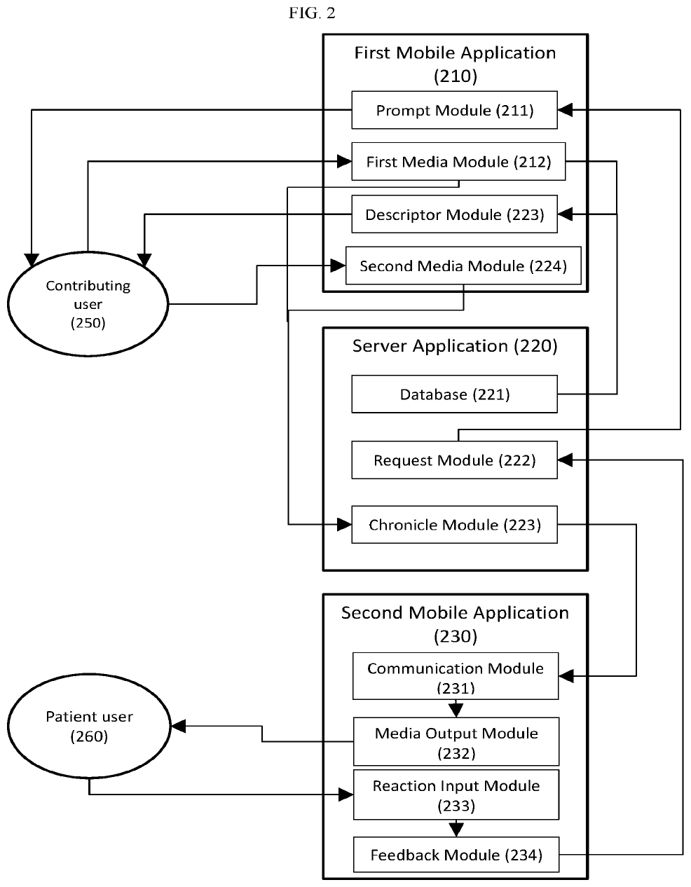

FIG. 2 shows a schematic diagram of a second exemplary reminiscence therapy arid media sharing platform fora patient user comprising: a contributing user mobile processor configured tn provide a first mobile application 210, a server processor configured to provide a server application 220.

In some embodiments, the first mobile application 210 comprises a prompt module 211, a first media module 212, a descriptor module 223,and a second media module 224. In some embodiments, the first mobile application 210 receives and displays a request for a first media from a contributing user 250. In some embodiments, the first media module 212 receives the first media from the contributing user 250. In some embodiments, the descriptor module 223 notifies the contributing user 250 to submit a second media based on the first media and a template. In some embodiments, the second media module receives the second media from the contributing user 250.

In some embodiments, the server application 220 comprises a database 221 a request module, 222, and a chronicle module 223. In some embodiments, the database 221 stores a plurality of the templates. In some embodiments, the request module 222 generates and/or submits the request. In some embodiments, the chronicle module 223 receives at least one of the first media and the second media. In some embodiments, the chronicle module 223 further generates a story based at least on the first media and a second media.

In some embodiments, the second mobile application 230 comprises a communications module 231, a media output module 232, and a reaction module 233, and a feedback module 234. In some embodiments, the communications module 231 receives the story. In some embodiments, the media output module 232 presents the story to a patient user 260. In some embodiments, the reaction module 233 measures a reaction of the patient user 260. In some embodiments, the reaction module 233 measures a reaction of the patient user 260. In some embodiments, the reaction module 233 measures a reaction of the patient user 260 while media output module 232 presents the story to a patient user 260. In some embodiments, the feedback module 234 transmits the reaction to the request module 222. In some embodiments, the request module 222 generates the request based on the reaction.

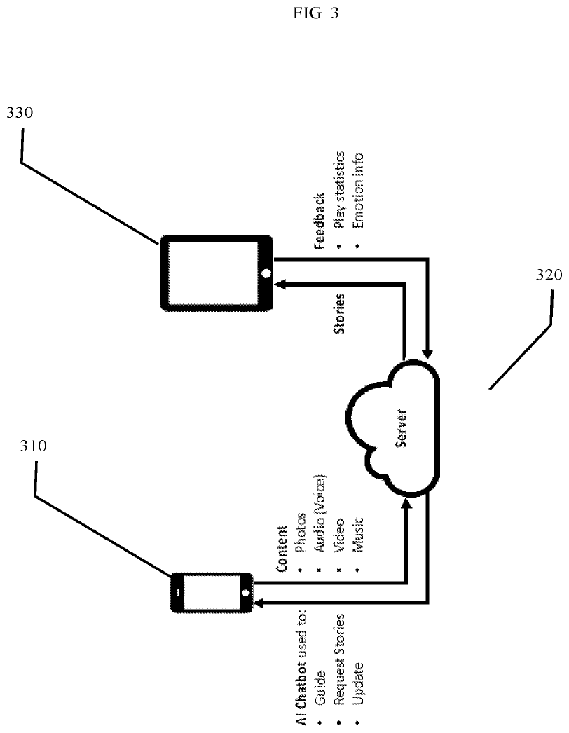

FIG. 3 shows a schematic diagram of a third exemplary reminiscence therapy and media sharing platform for a patient user comprising a contributing user application on a contributing user device 310, a server 320, and a patient user application on a patient user device 310. In some embodiments, the third exemplary reminiscence therapy and media sharing platform for a patient user transmits stories comprising media from the contributing user to the patient user.

The server 320 may guide, request stories, and or provide updates to the contributing user device 310 through an AI chatbot. The AI chatbot may comprise, a text message, a push notification, a call, an email, or any combination thereof. The server 320 may guide and/or request stories based on a theme. The AI chatbot may comprise a conversational goal-seeking tool that employs natural language conversation, to prompt user communication and additional submission of media. The chatbot may request information from the contributing, user based on the requirements of the patient and/or based on what stories need to be completed. In some embodiments, the AI chatbot comprises a mobile browser card. The mobile browser card may comprise a text message with instructions to provide the second media. The mobile browser card text message may comprise an external link to submit the second media. The external link may be a link to a mobile browser page. The mobile browser page may comprise buttons, text boxes, sample answers, microphone inputs, or any combination thereof to receive the second media.

In some embodiments, the AI chatbot comprises a web application that allows family to upload the first media, upload the second media, edit stories, or any combination thereof. The web application AI chatbot may be the same as the mobile browser card link. The web application may comprise buttons, text boxes, sample answers, microphone inputs, or any combination thereof to receive the second media.

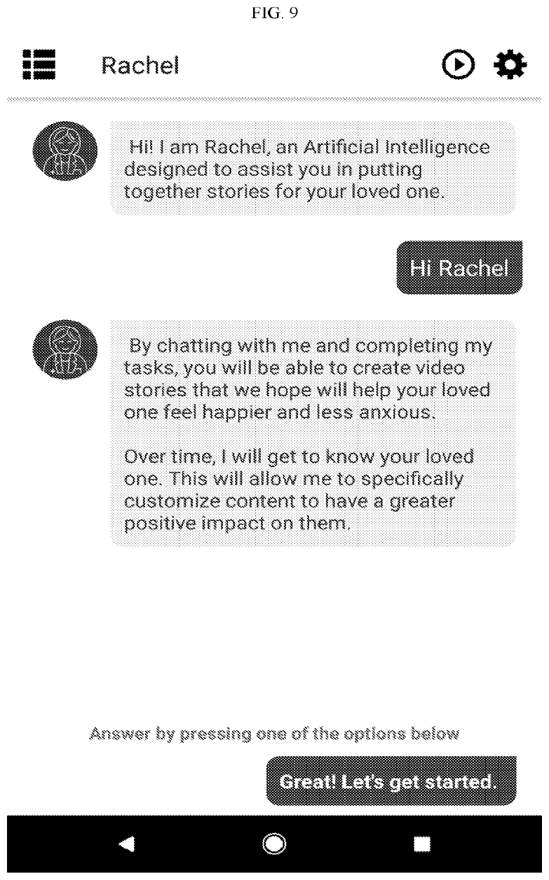

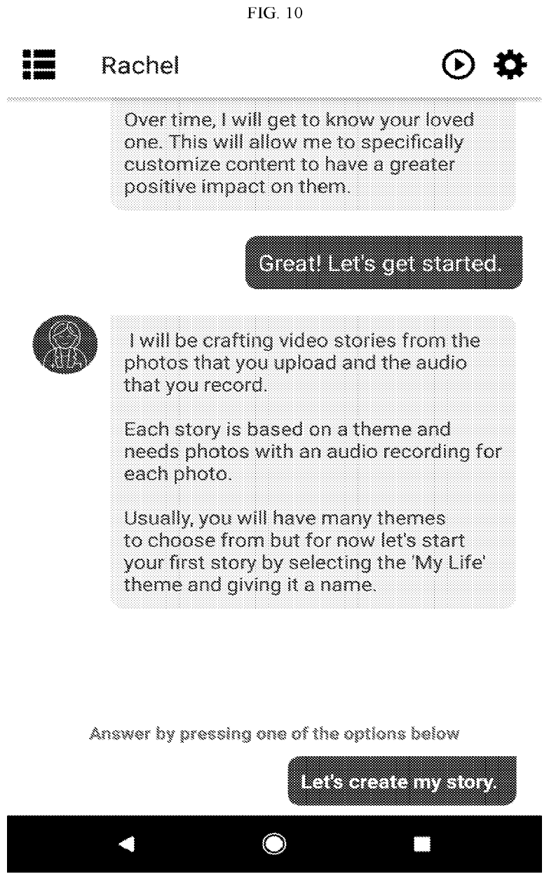

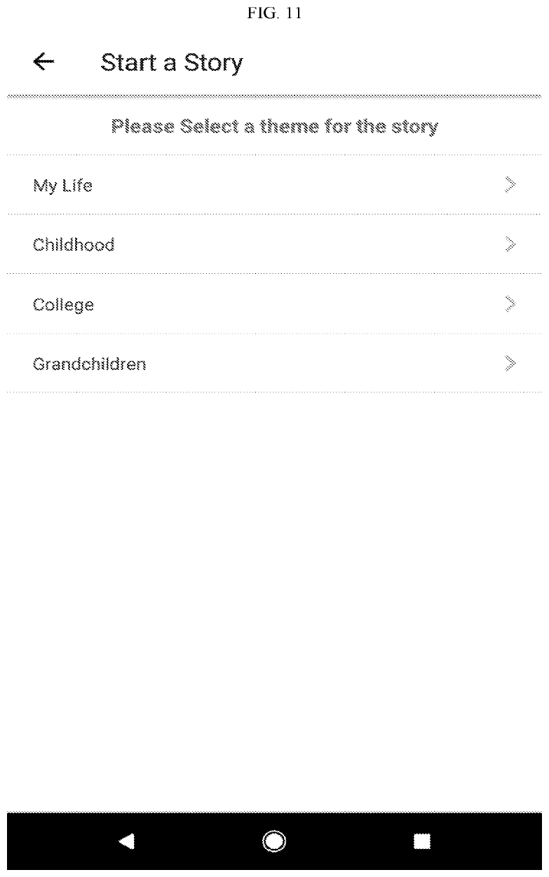

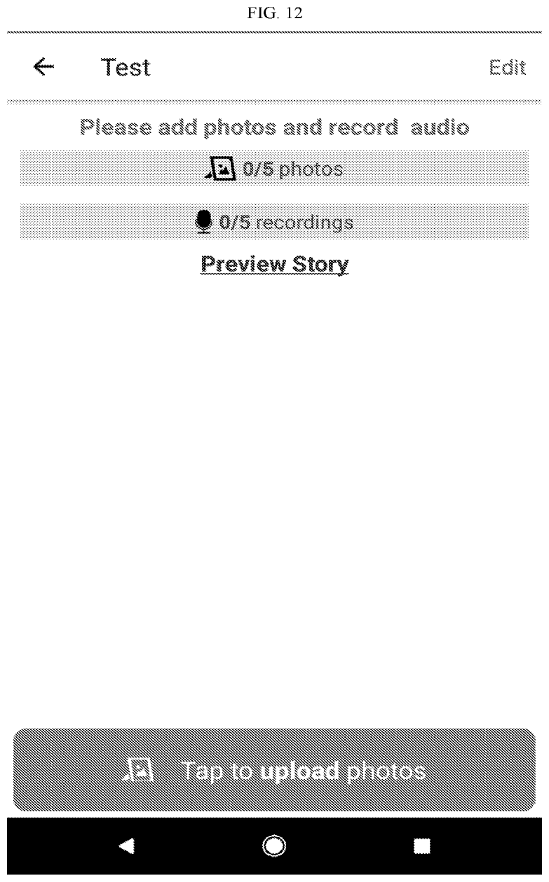

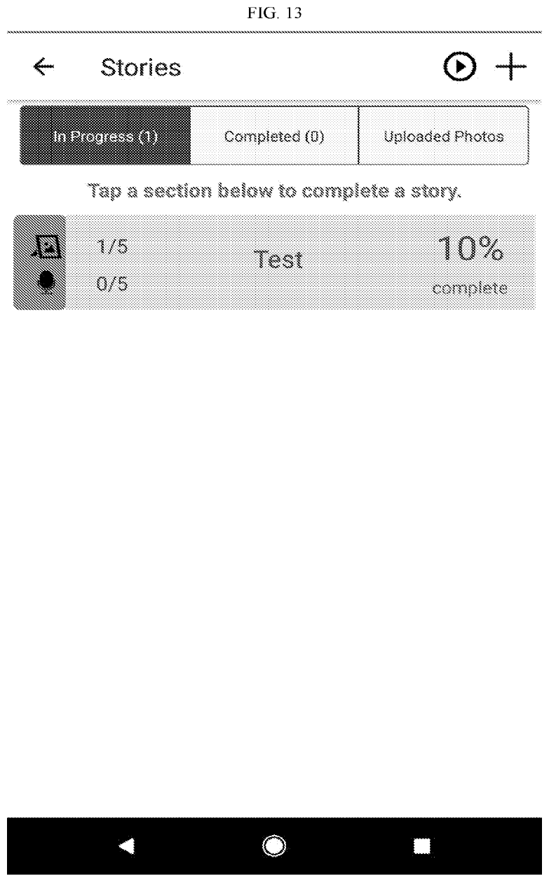

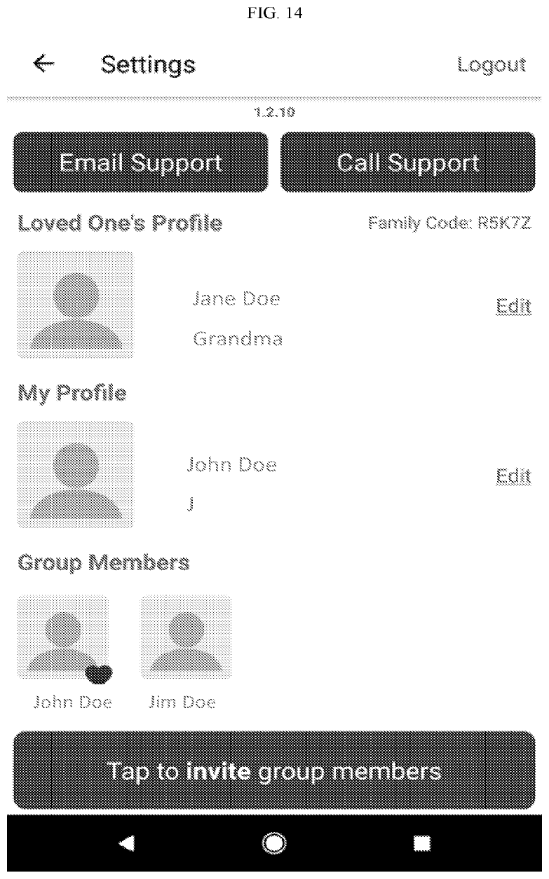

In some embodiments, the story comprises a documentary-style story. The theme may comprise, for example, a wedding, a vacation, and a birthday. The contributing user device 310 may then transmit a content comprising a photo, an audio file, a video, music, or any combination thereof to the server 320. The contributing user device 310 may then record the content openly, or through a scripted speech. Exemplary themes and theme related prompts are shown in FIGS. 9-11.

The server 320 may then transmit the content to the patient user device 310 in the form a story. The story may comprise a script comprising the content in a certain simultaneous or consecutive order. In some embodiments, the patient user device 310 displays the story to the patient user on demand, periodically, repetitively, or any combination thereof. In some embodiments, the server 320 or the contributing user determines the simultaneous or consecutive order, the periodicity, the repetition, or any combination thereof. The patient user device 310 may then collect feedback comprising play statistics and/or emotional information to the server 320. The server 320 may guide, request stories, and or provide updates to the contributing user device 310 based on the feedback.

In some embodiments the server 320 comprises a cloud based server. In some embodiments the server 320 comprises Amazon Web Services (AWS). User Devices

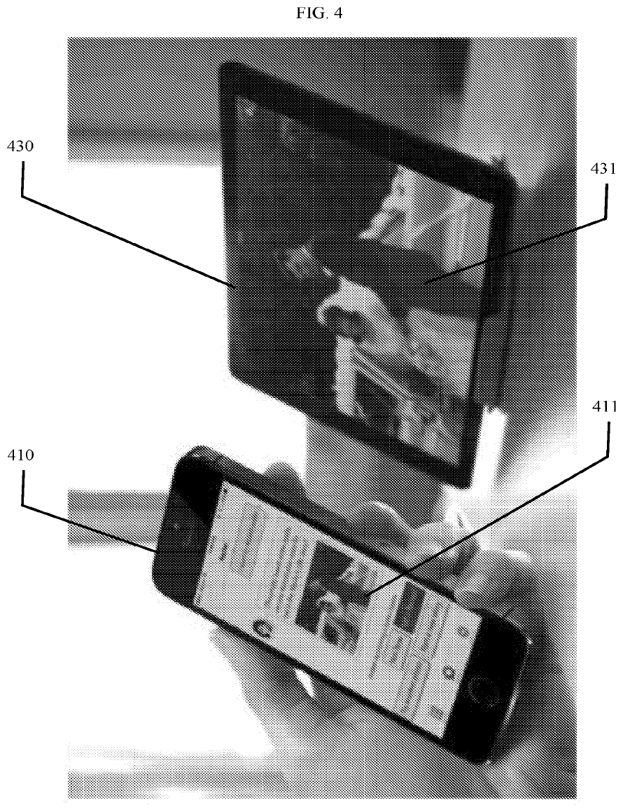

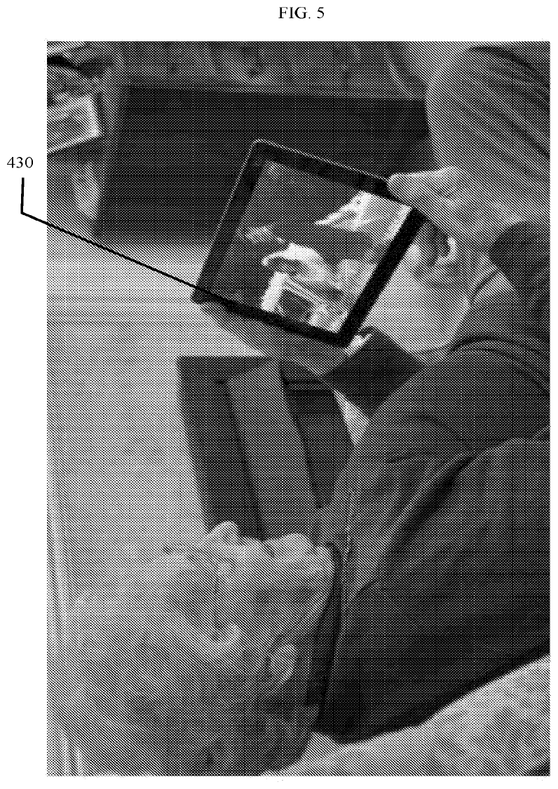

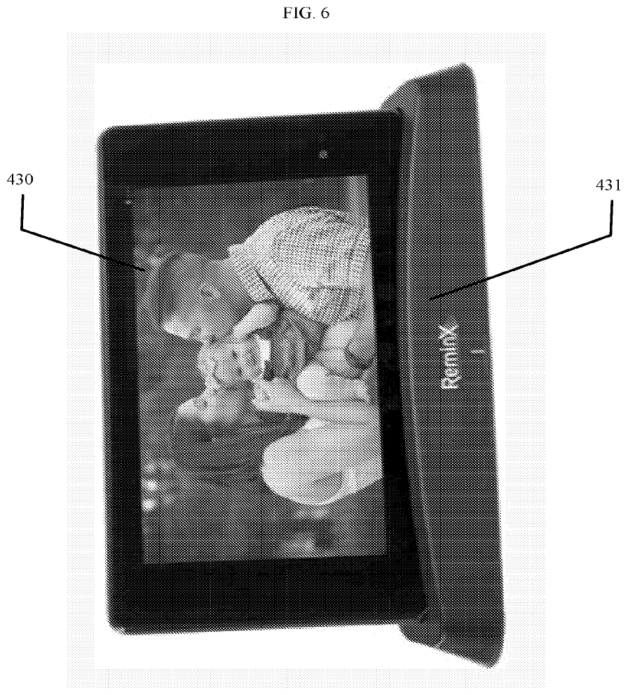

FIGS. 4-6 show an exemplary contributing user device, an exemplary patient user device, an exemplary first media, and an exemplary story. In some cases, the contributing user mobile processor configured to provide a first mobile application exists on a contributing user device 410. In some cases, the patient user mobile processor configured to provide a second mobile application exists on a patient user device 420. In some embodiments, the first mobile application receives the first media 411. In some embodiments, the second mobile application displays a story 431 based on the first media 411.

In some embodiments, at least one of the contributing user device 410 and the patient user device 420 comprises a desktop computer, a laptop computer, a notebook computer, a sub-notebook computer a netbook computer, a netpad computer, a set-top computer, a media streaming device, a handheld computer, an Internet appliance, a mobile smartphone, a tablet computer, a personal digital assistant, a video game console, a vehicle, or any combination thereof.

### Treatment Metrics

An exemplary test examined the impact of the platform, systems, and methods herein towards in-home use in patients with mild to moderate dementia. The 14 outpatient patients were studied who were 60 years or older, were diagnosed with dementia, displayed symptoms of mild to moderate cognitive deficits based on an MDRS total score of no less than 110, and exhibited adequate hearing, vision, and comprehension of English.

Each patient first responded to a patient questionnaire to assess their levels of depression, anxiety, and overall emotional distress. Each patient's caregiver rated the patient's level of emotional functioning using a caregiver-based questionnaire. Thereafter, on the same day, patients and their caregivers were instructed on how to use the reminiscence therapy and media sharing platform for a patient user. Patients then viewed the stories and were immediately re-assessed using the patient questionnaire.

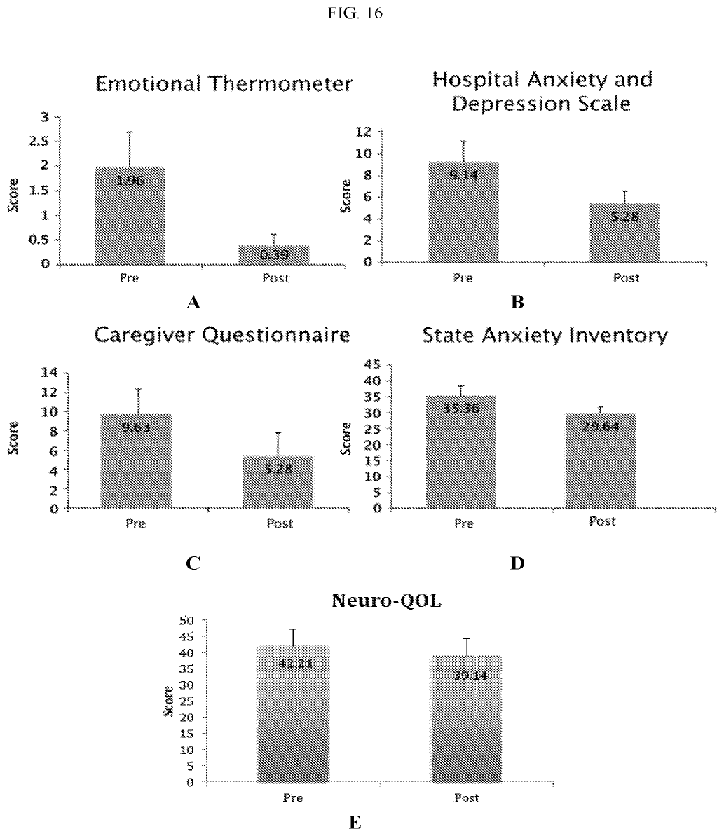

FIGS. 16A-16E show average patient user evaluation metrics before and after media rehabilitation treatment. Greater scores on each test indicate greater levels of emotional distress. Error bars are represented as Standard Error of the Mean (SEM).

Per FIG. 16A shows exemplary measurement results of an emotional thermometer (ET) test. In this example, the ET pre-viewing score was 1.96 and the post-viewing score was 0.39. FIG. 16B shows exemplary measurement results of a State Anxiety Inventory (STAI) test. In this example, the STAI pre-viewing score was 35.36 and the post-viewing score was 29.64. FIG. 16C shows exemplary measurement results of a Hospital Anxiety and Depression Scale (HADS) test. In this example, the HADS pre-viewing score was 9.14 and the post-viewing score was 5.28. FIG. 16D shows exemplary measurement results of a Neuro-Quality of Lite (NQOL) rest. In this example, the NQOL pre-viewing score was 42.21 and the post-viewing score was 39.14. FIG. 16E shows exemplary measurement results of a Caregiver Questionnaire (CQ) test. In this example, the CQ previewing score was 9.63 and the post-viewing score was 3.28.

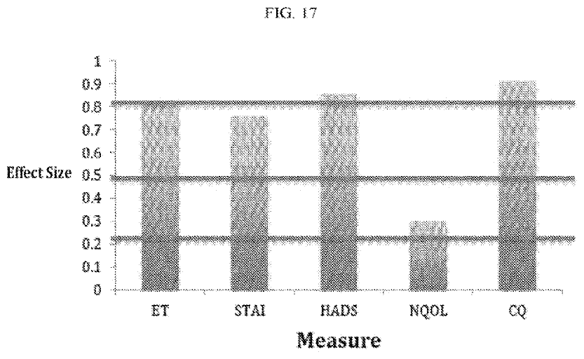

Per FIG. 16A and FIG. 17, which shows the effect sizes for the exemplary measurements in FIGS. 16A-16E, patients reported significantly less anxiety, depression, and overall emotional distress after having viewed their story. Effect sizes are used to quantify the magnitude of a statistical effect, with 0.20 typically being viewed as a “small” effect size. 0.50 being viewed as a “moderate” effect and 0.80 being considered as a “large” effect size. Effect sizes may be computed using a Morris and DeShon's method. The large effect sizes displayed by these exemplary results were about 0.76 to 0.91.

Various additional evaluation metrics can be used to assess behavioral symptoms, such as anxiety, apathy, agitation, depression, as well as quality of life and caregiver burden.

As such, the reminiscence therapy and media sharing platform for a patient user provided herein has an immediate and positive impact on emotional functioning in patients with dementia. These positive results coupled with the increased accessibility and case of use of the platforms, systems, and devices herein highlights the important impact of the disclosure herein.

### Digital Processing Device

In some embodiments, the platforms, systems, media, and methods described herein include a digital processing device, or use of the same. In further embodiments, the digital processing device includes one or more hardware central processing units (CPETs) or general purpose graphics processing units (GPGPETs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non -limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non limiting, examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxce®, Google TV®, Google Chromecast®, Amazon Fire®, and Samsung® HomeSync®. Those of skill in the art will also recognize that suitable video game console operating systems include, by way of nun-limiting examples, Sony® PS3®-Sony® PS4®, Microsoft® Xbox 360®, Microsoft Xbox One, Nintendo® Wii Nintendo® Wi i U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing, device, such as a VR headset. In further embodiments, suitable YR headsets include, by way of non-limiting examples, HTC Vive, Oculus Rift, Samsung Gear YR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In same embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

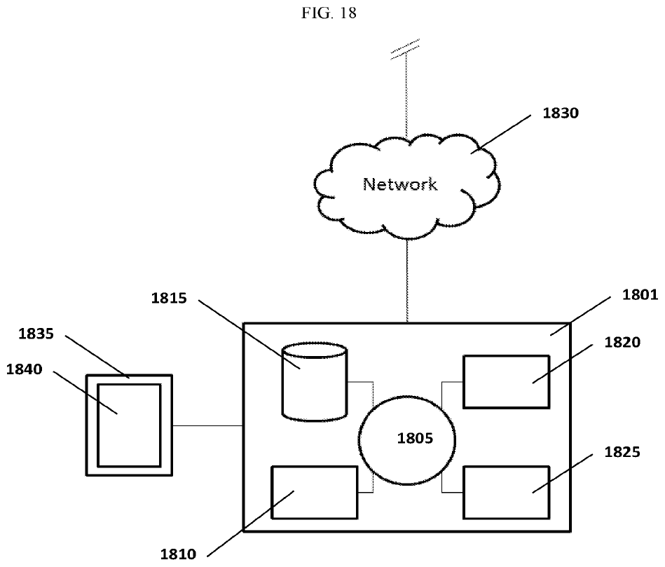

Referring to FIG. 18, in a particular embodiment, a digital processing device 1801 is programmed or otherwise configured to provide a reminiscence therapy application. The device 1801 is programmed or otherwise configured to provide a reminiscence therapy application. In this embodiment, the digital processing device 1801 includes a central processing unit (CPU, also “processor” and “computer processor” herein) 1805, which is optionally a single core, a multi core processor, or a plurality of processors for parallel processing. The digital processing device 1801 also includes memory or memory location 1810 (e.g., random-access memory, read-only memory, flash memory), electronic storage unit 1815 (e.g., hard disk), communication interface 1820 (e.g., network adapter) for communicating with one or more other systems, and peripheral devices 1825, such as cache, other memory, data storage and/or electronic display adapters. The memory 1810, storage unit 1815, interface 1820 and peripheral devices 1825 are in communication with the CPU 1805 through a communication bus (solid lines), such as a motherboard. The storage unit 1815 comprises a data storage unit (or data repository) for storing data. The digital processing device 1801 is optionally operatively coupled to a computer network (“network”) 1830 with the aid of the communication interface 1820. The network 1830, in various cases, is the internet, an internet, and/or extranet, or an intranet and/or extranet that is in communication with the internet. The network 1830. In some cases, is a telecommunication and/or data network. The network 1830 optionally includes one or more computer servers, which enable distributed computing, such as cloud computing. The network 1830, in some cases, with the aid of the device 1801, implements a peer-to-peer network, which enables devices coupled to the device 1801 to behave as a client or a server.

Continuing to refer to FIG. 18. the CPU 1805 is configured to execute a sequence of machine-readable instructions, embodied in a program, application, and/or software. The instructions are optionally stored in a memory location, such as the memory 1810. The instructions are directed to the CPU 105, which subsequently program or otherwise configure the CPU 1805 to implement methods of the present disclosure. Examples of operations performed by the CPU 1805 include fetch, decode, execute, and write back. The CPU 1805 is, in some cases, part of a circuit, such as an integrated circuit. One or more other components of the device 1801 arc optionally included in the circuit. In some cases, the circuit is an application specific integrated circuit (ASIC) or a field programmable gate array (FPGA).

Continuing to refer to FIG. 18. the storage unit 1815 optionally stores files, such as drivers, libraries and saved programs. The storage unit 1815 optionally stores user data, e.g., user preferences and user programs. The digital processing device 1801, in some cases, includes one or more additional data storage units that are external, such as located on a remote server that is in communication through an intranet or the internet.

Continuing to refer to FIG. 18, the digital processing device 1801 optionally communicates with one or more remote computer systems through the network 1830. For instance, the device 1801 optionally communicates with a remote computer system of a user. Examples of remote computer systems include personal computers (e.g., portable PC). slate or tablet PCs (e.g., Apple® iPad, Samsung® Galaxy Tab. etc.), smartphones (e.g., Apple® iPhone, Android-enabled device, Blackberry®, etc.), or personal digital assistants.

Methods as described herein are optionally implemented by way of machine (e.g., computer processor) executable code stored on an electronic storage location of the digital processing device 101, such as, for example. on the memory 1810 or electronic storage unit 1815. The machine executable or machine readable code is optionally provided in the form of software. During use, the code is executed by the processor 1805. In some cases, the code is retrieved from the storage unit 1815 and stored on the memory 1810 for ready access by the processor 1805. In some situations, the electronic storage unit 18I5 is precluded, and machine-executable instructions are stored on the memory 1810. Non-transitory computer readable storage medium

In some embodiments, the platforms, systems media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects. Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR), In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, serverside coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk. WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

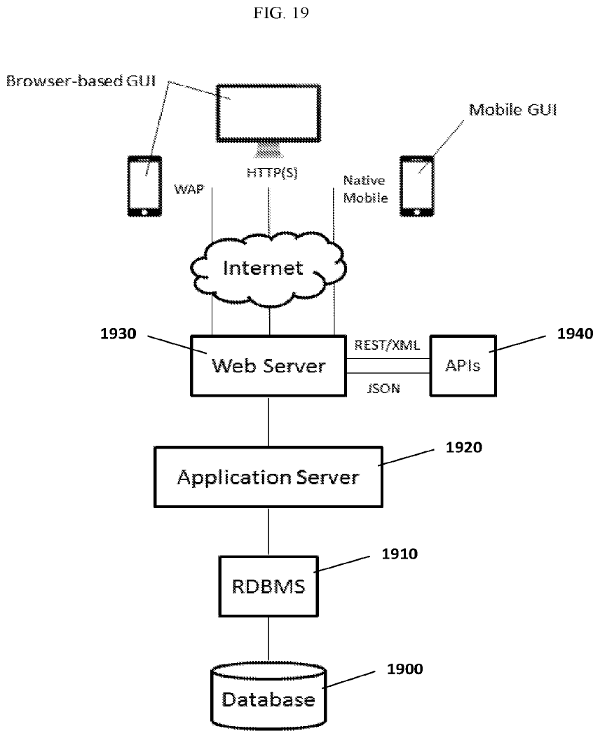

Referring to FIG. 19, in a particular embodiment, an application provision system comprises one or more databases 1900 accessed by a relational database management system (RDBMS) 1910. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 1920 (such as Java servers, .NET servers, PHP servers, and the like) and one or more web servers 1930 (such as Apache, IIS, GWS and the like). The web server(s) optionally expose one or more web services via app application programming interfaces (APIs) 1940. Via a network, such as the Internet, the system provides browser-based and/or mobile native user interfaces.

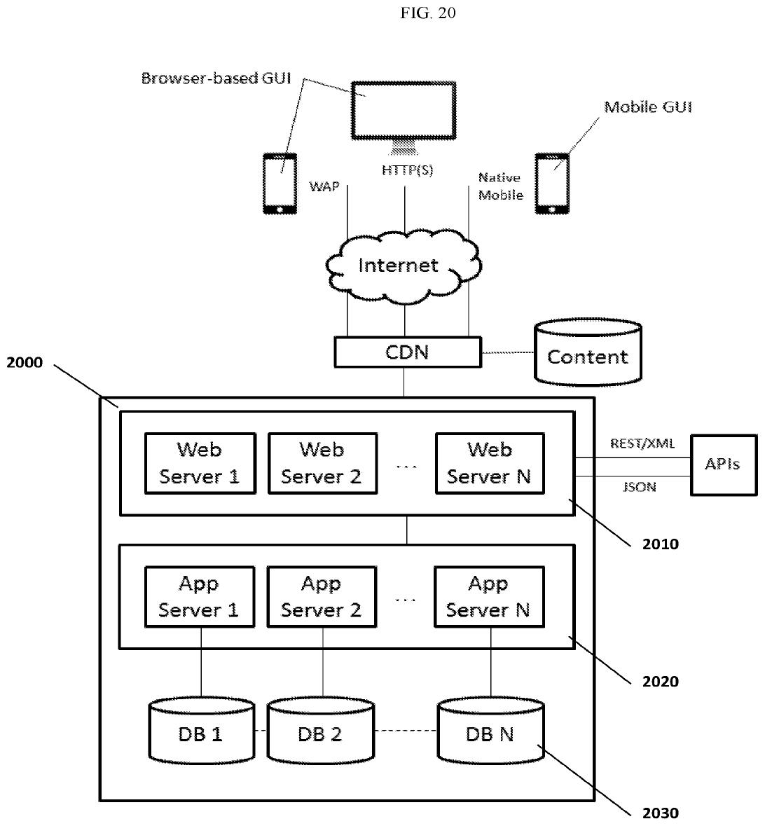

Referring to FIG. 20, in a particular embodiment, an application provision system alternatively has a distributed, cloud-based architecture 2000 and comprises elastically load balanced, auto-scaling web server resources 2010 and application server resources 2020 as well synchronously replicated databases 2030.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Javascript™, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appeelerator®, Celsius, Bedrock, Flash Lite, .NET Compact Framework, Rhomobile, and Worklight Mobile Platform. Other development environments are available without cost including, by way of non-limiting examples, Lazarus, MobiFlex, MoSyne, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Swift, Kotlin, Javascript™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET. or combinations thereof.

Web browsers (also called Internet browsers) are software applications, designed for use with network-connected digital processing devices, for retrieving, presenting, and traversing information resources on the World Wide Web. Suitable web browsers include, by way of non limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser, Mobile web browsers (also called mircrobrowsers, mini-browsers, and wireless browsers) are designed for use on mobile digital processing devices including, by way of nonlimiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Black Berry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for providing a reminiscence therapy application. In various embodiments, suitable databases include, by way of non-limiting examples, relational databases, non-relational databases, object oriented databases, object databases, entity relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Testing Interface

An artificial intelligence (AI) chatbot interacts with family, friends, and other caregivers, encouraging and reminding them to provide photos, voice narration, video content, and music selections, and organizes the content into personalized stories for their loved one. The AI chatbot is a conversational goal-seeking tool, using natural language conversation, that understands the requirements of the end user (senior loved one) and tries to get the family to complete tasks that satisfy the requirements. The AI chatbot is intended specifically to make it easy to contribute content with minimal investment of time and effort on the part of the family.

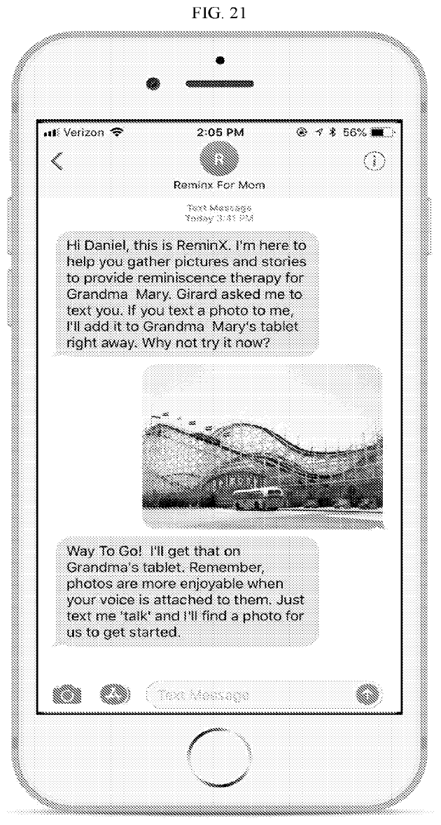

Referring to FIG. 21, interaction with the product by content contributors such as family caregivers is primarily driven through text message communication. Each family is given a private concierge phone number that is shared among the family. The AI chatbot guides family members to text photos or videos to the concierge number as well as to narrate stories over the photos via voicemail. The content then appears automatically on the end user's Tablet. The concierge phone number can be given to the customer's family and friends so they can also contribute content and collaborate in the care of the end user.

In addition to text messaging, the product has several additional channels of content collection. These include the a contributor mobile app (i OS/Android), a contributor web app, and email. These modalities duplicate many of the functions that are available via text message. providing caregivers with multiple options to contribute and organize content, depending on their preference.

The AI can automatically reach out to contributors via text messaging at a specified time interval (e.g., once per week, once per day) to encourage contributors to provide photos and videos and voice recordings over previously uploaded content to further optimize content as needed by the AI. The AI can also provide short messages with encouragement and instruction that facilitates additional content curation, or it can provide updates on the status of the patient, or updates on how the patient responded to specific content.

### Fast-Story Creation

In order to rapidly generate content onto a tablet, an administrative tool allows administrative users to add semi-personalized content to the tablet. Questions can be asked of family members or caregivers, such as: “Where did your loved one grow up? What are your loved one's favorite sports teams? Who is your loved one's favorite actor/actress?.

This information is used to create curated stories based on generic interests and memories (i.e., 1950s Chicago; 1962 Dodgers) specially for that patient's tablet, even if personal content is limited. This way the tablet can be shipped with pre-loaded stories that are personalized for the patient, and the product can be used immediately upon delivery even if the family has not yet begun to upload content.

### Terms and Definitions

Unless otherwise defined, all technical terms used herein have the same meaning as commonly understood by one of ordinary skill in the art to which this disclosure belongs.

As used herein, the singular forms “a, ” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

### EXAMPLES

The following illustrative examples are representative of embodiments of the software applications, systems and methods described herein and are not meant to be limiting in any way.

### Example 1

Joanne is the primary at-home caregiver of her father, who has middle-stage Alzheimer's disease. After showing progressing signs of agitated behavior, her father's doctor suggests that he receives reminiscence therapy. After meeting with the doctor, Joanne decides to order the digital reminiscence therapy and media sharing platform.

A few days later, a package arrives at her door. Joanne opens up the box to find the reminiscence therapy tablet, a charging station, the charging cord, an instructional pamphlet, and a welcome letter which includes a family code. Following the instructions, Joanne downloads the reminiscence therapy family app and creates an account with the family code provided. After sliding through the in-app informational onboarding. Joanne receives a message from the family app's artificial intelligence bot.

The artificial intelligence bot guides Joanne through the process of creating a story for her father. Joanne picks a theme for the story, then is prompted by the artificial intelligence bot to upload a photo. Joanne looks through her photos and finds an old picture of her father that fits the theme of her story. She uploads the photo, then is prompted again to record a narrative audio that describes the picture. The narrative audio is intended to provide a personal message from a familiar voice that describes the people in the photos, the year, and the setting.

The artificial intelligence bot also asks Joanne to select music that her father likes. Individual songs, artists, or genres can be selected. The music will be played over the stories as her father watches them, and the tempo of the music can be automatically varied depending on the time of day. For example, faster tempo music can be played in the morning and slower tempo music can be played prior to bed or during sundowning.

When Joanne completes her task, the intelligence bot gives her the option to create another story or to complete the one she is currently working on. Over the next few minutes, Joanne uploads enough pictures and videos and records enough audio narration to finally complete her first story, at her convenience. When the story is completed, Joanne is congratulated by the artificial intelligence bot and as a reward, receives a link to an article on how at-home caregivers can practice self-care.

The following afternoon, when Joanne's father begins “sundowning” and begins to display agitated behaviors, she starts to set up the tablet, Joanne connects the charging cord to the dock, then plugs it into an outlet. She turns on the tablet and is prompted to connect it to the wifi. Immediately after Joanne enters her wifi password, the content she uploaded from the family app is loaded on to the reminiscence therapy tablet, and the tablet starts to play the story that she had previously created with the artificial intelligence bot. Joanne sets the tablet down in front of her father and allows him to watch the story she created. Her father becomes very engaged in the reminiscence content.

The reminiscence content causes Joanne's father to access old memories in his brain using his remote memory. Seniors are known to adapt to aging by using familiar knowledge, skills, and strategies to develop stable patterns of activity. Remote memory, within includes reminiscence processes, is typically the last system to deteriorate in the elderly. Increased use of remote memory in older adults is known to improve general cognitive function.

Later that night, Joanne receives a message that her father had smiled 3 times during his first digital reminiscence therapy session. lncentivized by the feedback, Joanne invites more people in her family to contribute to adding media to her father's tablet.

The next day, the artificial intelligence bot asks Joanne questions about her father's interest in sports. Joanne responds back with the name of her father's favorite football team. The bot informs Joanne that it will be creating a story of that football team and sends her a link to preview the story. Later that day, Joanne views the story and gives the artificial intelligence bot her approval. As Joanne's father is watching the digital reminiscence therapy tablet, she notices that a new story of her father's favorite football team was automatically uploaded to his tablet amongst the other stories she and other group members have created.

At the end of the week, Joanne receives a notification that displays her father's usage of the reminiscence therapy tablet. The notification message includes the cumulative time her father spent watching stories, his total number of smiles, and which story he enjoyed the most.

### Example 2

Susan has been diagnosed with severe Alzheimer's disease. She requires constant care and is living in a memory care facility. Her psychiatrist notices that she is not responding to her Alzheimer's medications and that she is exhibiting, cognitive decline, depression, and communication difficulties. She is prescribed the reminiscence therapy tablet as a treatment for her symptoms, since studies have shown activation of remote memory to reduce depression and improve communication.

The nursing staff at the memory care facility have several units of the reminiscence therapy tablet, which includes a tablet, charging, station, the charging cord, an instructional pamphlet, and a welcome letter. The nursing staff also has a center administrative portal where they manage all of the reminiscence therapy units for all of their patients that use the device. The setup Susan's account using a new family code and they enter certain preferences into the portal. For example, they know that Susan likes impressionist art, gardening, hiking, and sewing. They also enter in Susan's age.

The tablet automatically generates “pre-created” story content based on Susan's age and preferences. Because her peak memory years were in the 1950s, the pre-created stories focus on creating stories with images and videos of impressionist art, gardening, hiking, and sewing that may have been popular in the 1950s. This way there is some baseline reminiscence material on the tablet.

The nursing staff also contacts Susan's son, Henry, who is most actively involved in Susan's care. They explain the reminiscence therapy tablet to Henry and give him the family code so that he can download the family app. When it is finished downloading, he opens the app and goes through the in-app informational onboarding. He then immediately invites his other family members to the digital reminiscence therapy and media sharing platform.

Henry receives his first message from the family apps artificial intelligence bot. The artificial intelligence bot introduces itself, then guides Henry through the process of creating a story for his mother. In instructs Henry to focus on old photos and content from Susan's peak memory years, since she is unlikely to remember recent events. After Henry picks a general theme for the story, the bot prompts hint to upload a photo and to add narrative audio to describe it. W hen Henry completes that task, the artificial intelligence bot gives him the option to continue with that story, or to start a new one. Henry decides he will continue working on the digital reminiscence therapy and media sharing platform tomorrow.

The following day, Henry receives a notification from the artificial intelligence bot to continue uploading more photos for his mother. When he opens the family app, he notices that other group members have already created stories as well. The stories tell his mother's past. He then follows along with the artificial intelligence bot's instructions and uploads enough photos and narrative audio until his first story is completed. As a reward, the bot sends Henry links to articles that inform him about severe Alzheimer's dementia.

Later that week, the nurses setup the tablet in Susan's room and give let her view the stories that were created by the family. The nurses immediately notice changes in Susan's mood as she continuously points to people on the reminiscence tablet and begins to tell positive stories about her experiences. Because she is so engaged in the content and because the tablet is so easy to use, the nurses are able to temporarily leave the room and allow Susan to watch the content on her own for 10 more minutes.

The next day, Henry receives messages from the artificial intelligence bot to continue uploading media and receives questions about his mother. The nurses have indicated that Susan refuses to take showers. The artificial intelligence bot asks Henry to record an audio message telling his mother how important it is to take showers. On shower days, the nursing staff plays the audio message to Susan to motivate her to get our of bed and take a shower.

Once a week, the memory care facility has group activity sessions that feature the reminiscence therapy tablet. Groups of five dementia patients sit together and talk about their stories, while holding the tablet in their hand. All five patients communicate much more than they normally do.

At the end of the week, Henry receives a message with a summary of his mother's usage of the reminiscence therapy tablet. The notification includes the cumulative time his mother spent watching stories, her total number of smiles, and the stories that she enjoyed the most.

After using the tablet on a daily basis for 2 weeks, the nursing staff starts to notice that Susan is less depressed and more communicative. When Henry comes back to visit his mother he also notices improvements in her mood, cognitive function, and communication.

### Example 3

Margaret is an 87-year old woman that lives by herself in a rural area, where her nearest neighbors are at least a few miles away. She used to take trips to town often. However, now she only goes when she is in need of necessities, due to the long distance. While Margaret's family visits her as often as they can, she spends the majority of her days in isolation since her three children ended up settling in their college towns on the coast. Margaret calls them on the phone once a week, however her children's busy schedules only allow her to talk to them for a few minutes at a time. In a recent call, her children have noticed an increase in her depressive symptoms and lack of emotion due to social isolation.

One day, her eldest son calls Margaret and informs her that she should be expecting a package from him soon. The package finally arrives a few days later, and Margaret opens it to find the reminiscence therapy tablet, the charging station, the charging cord, an instructional pamphlet, and a welcome letter. Following the simple instructions in the pamphlet, Margaret connects the charging cord to the charging station and plugs it into the wall on her bedside table. She turns on the tablet and it prompts her to enter her wifi code.

Immediately after she enters the code, the reminiscence tablet begins to display photos and narrative audio. She watches the tablet as it shows old pictures of her family—her husband who had passed away, and her children when they were very young involved in a variety of different activities. She listens to her children's recorded narration as she watches, and they remind her of all the great times they have had. As she continues watching, the photos start to become more recent. She watches a video of her granddaughter playing soccer last week and views pictures of her children with their growing families. When Margaret reaches the end of her stories, she places the reminiscence tablet back on the charging station.

The next day, when Margaret picks up the reminiscence tablet, she finds that there are new pictures and videos for her to watch. Margaret watches a video of her grandson walking for the first time while she listens to the audio of her daughter telling her all about her experience. Every day, there are new photos or videos of her family for Margaret to watch, ranging from special events to everyday moments.

In the next couple days, Margaret picks up the tablet and it begins to play videos of her children and grandchildren wishing her a happy Mother's Day. Each of her children had sent her a video with their families for Mother's Day. In the evening, Margaret's eldest son calls her and notes that his mother sounds happier and is more talkative. When Margaret's son hangs up, he uploads more media to the family app and reminds his siblings to do the same.

### Example 4

Juan recently had a stroke. He receives the reminiscence therapy tablet to help motivate him and educate him on how to more rapidly recover from stroke. His family uses the texting interface to upload content and narrate stories over older photos of times when he was active, productive, and happy. The family was given a shared phone number and share a text messaging thread using that phone number where they can text photos that appear on Juan's reminiscence therapy tablet. An artificial intelligence chatbot communicates with the family members through the text messaging thread and asks them to upload photos and videos. It also asks them to narrate stories over photos by leaving voicemails on that phone number. If they agree with the chatbot's request to leave a voicemail over a specific photo. the chatbot calls them back and the call goes straight to voicemail, and they tell the story of the photo in the voicemail. The voicemail is then recorded and linked to the photo and both are presented together on the tablet. The family can also perform additional functions through the text message interface or by accessing the mobile reminiscence therapy website through the text messaging interface. The text messaging interface makes it very easy for the family to upload content onto Juan's tablet.

This content, and the fact that his family shows that they care for him, motivates Juan to try harder to perform rehabilitation exercises, which then expedites recovery from stroke. The tablet also provides educational content to Juan that helps him to understand what he needs to do, and provides messages from his physician that provides further instruction. His family also receives messages via the family app that educate them on how to best help Juan. Juan's physician is able to track his progress through biofeedback from the camera, sensors, and buttons on the tablet.

### Example 5

Frank is undergoing chemotherapy. He is anxious about having cancer and is nervous about chemotherapy. He is given a reminiscence therapy tablet during chemotherapy infusion while he is in the chair. Frank likes the beach. The tablet shows videos of waves washing cancer away. This calms Frank during chemotherapy. His family uses the reminiscence therapy website on their laptop computers to upload and organize photo and video content, and also receive a concierge phone number so that they can text photos and audio messages of encouragement. Collectively, the content on the tablet reduces Frank's anxiety while he is receiving chemotherapy. Frank later tells the doctor that he has trouble sleeping at night, due to anxiety from having cancer. His doctor recommends that he take the reminiscence therapy tablet home with him and that he views it for 10 minutes each night before bed. This reduces his anxiety and he is better able to sleep. Due to his reduced anxiety and his improved sleep, the update of his chemotherapy drugs is mire effective and his quality of life during chemotherapy is better.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1. A reminiscence therapy and media sharing platform for a patient user comprising:
(a) a contributing user mobile processor configured to provide a first mobile application comprising:
(i) a prompt module receiving and displaying a request for a first media from a contributing user;
(ii) a first media module receiving the first media from the contributing user; and
(b) a server processor configured to provide a server application comprising:
(i) a request module generating the request and submitting the request to the prompt module; and
(ii) a chronicle module receiving the first media and generating a story based on the first media; and
(c) a patient user mobile processor configured to provide a second mobile application comprising:
(i) a communications module receiving the story;
(ii) a media output module presenting the story to the patient user;
(iii) a reaction module measuring a reaction of the patient user while the media output module presents the story to a patient user; and
(iv) a feedback module transmitting the reaction to the request module; wherein the request module generates a subsequent request based on the reaction.

2. The platform of claim 1, wherein at least one the first media and the story comprises a photograph, a video, an image, a text, or any combination thereof.

3. The platform of claim 1, wherein the request comprises a media type, a media theme, a media subject, a media color, a media date, a media duration, or any combination thereof.

4. The platform of claim 1, wherein the prompt module displays the request for the first media via a screen, a speakerphone, a phone call, a text message, a push notification, an email, or any combination thereof.

5. The platform of any one of claim 1, wherein the reaction comprises an anger parameter, a contempt parameter, a fear parameter, a happiness parameter, a surprise parameter, a sadness parameter, a symmetry parameter, an eye quadrant, an eye fixation time, an eye fixation duration, a button press, or any combination thereof.

6. The platform of claim 1, wherein the reaction does not comprise a button press.

7. The platform of claim 1, wherein the reaction module measures the reaction of the patient user through a facial expression recognition process.

8. The platform of claim 7, wherein the facial expression recognition process is configured for facial recognition of the patient user having an age of greater than about 50 years.

9. The platform of claim 7, wherein the facial expression recognition process comprises a computer learning process.

10. The platform of claim 1 wherein the contributing user comprises a plurality of contributing users.

11. The platform of claim 1, wherein the chronicle module generates the story by performing at least the following: (a) performing a facial recognition on the first media; (b) performing an object recognition on the first media; (c) determining a geographic location associated with the first media.

12. The platform of claim 11, wherein the facial expression recognition process comprises an age recognition, a sex recognition, an environment recognition, an object recognition, or any combination thereof.

13. The platform of claim 1, wherein the chronicle module further stores the story.

14. The platform of claim 1, wherein the server application further comprises a database storing a plurality of the templates.

15. The platform of claim 14, wherein the first mobile application further comprises a descriptor module notifying the contributing user to submit a second media based on the first media and a template.

16. The platform of claim 15, wherein the second media is further based on a third media.

17. The platform of claim 15, wherein the first mobile application further a second media module receiving the second media from the contributing user.

18. The platform of claim 15, wherein the chronicle module further generates the story based on the second media.

19. The platform of claim 15, wherein the second media comprises a photograph, a video, an image, a gif, an emoji, a text, or any combination thereof.

20. The platform of claim 1, wherein the server application further comprises a media agglomeration module determining and receiving a third media from a third media source.

21.-55. (canceled)
